(19) United States
(12) Patent Application Publication
(10) Pub. No.: US 2008/0234244 A1
(43) Pub. Date: Sep. 25, 2008

(54) CUCURBITACIN B AND USES THEREOF

(76) Inventors: Wei Dong Xie, Hong Kong (CN); Kwan Li, Hong Kong (CN); Edgar Shiu Lam Liu, Hong Kong (CN); Kee Hung Chu, Hong Kong (CN)

Correspondence Address: EVAN LAW GROUP LLC, 600 WEST JACKSON BLVD., SUITE 625, CHICAGO, IL, 60661, US

(21) Appl. No.: 12/051,461

(22) Filed: Mar. 19, 2008

Related U.S. Application Data
(60) Provisional application No. 60/919,088, filed on Mar. 19, 2007.

(51) Int. Cl. A61K 31/575; A61P 35/04

(52) U.S. Cl. 514/180

(57) ABSTRACT

The present invention relates to uses of cucurbitacins and compositions comprising cucurbitacin B. The present invention also relates to methods for preventing or treating various diseases and disorders by administering to a subject in need thereof cucurbitacin B. The invention also encompass methods of developing a therapeutic that comprises a cucurbitacin using the signaling molecules in the Ras-Raf-Mek-Elk-STAT3 pathway.

### 1. CROSS REFERENCE TO RELATED APPLICATIONS

This application claims priority to U.S. Provisional Application No. 60/919,088, entitled “Cucurbitacin B and Uses Thereof” and filed on Mar. 19, 2007, the contents of which are hereby incorporated herein by reference.

### 2. BACKGROUND OF THE INVENTION

Cancer is the second leading cause of death in the United States. In the US, cancer accounts for 1 in every 4 deaths. The American Cancer Society estimated that in 2007, there would be 1.44 million new cases of cancer and that cancer would cause 560,000 deaths. Current cancer therapy involves surgery, chemotherapy and/or radiation treatment to eradicate neoplastic cells in a patient (see, for example, Stockdale, 1998, “Principles of Cancer Patient Management”, in Scientific American: Medicine, vol. 3, Rubenstein and Federman, eds., Chapter 12, Sections IV and X). All of these approaches pose significant drawbacks for the patient. Almost all chemotherapeutic agents are toxic, and chemotherapy can cause significant, and often dangerous, side effects, including severe nausea, bone marrow depression, immunosuppression, etc. Additionally, many tumor cells are resistant or develop resistance to chemotherapeutic agents through multi-drug resistance. Therefore, there is a significant need for novel compounds and methods that are useful for treating cancer with increased specificity and reduced side effects.

Natural cucurbitacins are predominantly found in the family Cucurbitaceae which contain some 900 species in about 100 genera, many familiar as the wild gourds, squash, cucumbers, and melons of Cucurblta, Cucumis, Citrullus, Marah, Echinocystis, Lagenaria, Scyos, Ecballium, and Bryonia. At least 100 species in 30 genera have been shown to contain cucurbitacins—a group of oxygenated tetracyclic triterpenes that are responsible for the characteristic bitter taste of most wild Cucurbitaceae.

Cucurbitacin B can be extracted from a traditional Chinese medicine, namely the stem-end of Cucumis melo L. It is used traditionally to treat hepatitis and liver cancer. Recent findings also indicated that cucurbitacin B protects against CCl4-induced hepatotoxicity (Agil et al., Planta Med. 1999; 65:673-5) and bear anti-cancer and anti-inflammatory activities (Jayaprakasam et al., Cancer Lett. 2003; 189:11-6).

However, the mechanism of the action of cucurbitacin B is unknown so far. It is reported to exert its toxicity on cancer cells by disruption of the actin cytoskeleton (Rabow et al., J Med. Chem. 2002; 45:818-40), which is also a feature characterized by its related compound, cucurbitacin E and cucurbitacin glucoside (Duncan et al., Biochem Pharmacol. 1996; 52:1553-60; and Tannin-Spitz et al., Biochem Pharmacol. 2007; 73:56-67.).

The present invention relates to an investigation of the mechanism of cucurbitacin B activity and provides the use of cucurbitacin B to treat or prevent certain cancers. Citation of any reference in Section 2 of this application is not to be construed as an admission that such reference is prior art to the present application.

### 3. SUMMARY OF THE INVENTION

The present invention provides methods of using cucurbitacin B in the prevention, treatment or management of cancers. The present invention provides novel compositions comprising cucurbitacin B, such as but not limited to dietary supplements, food additives, and pharmaceutical compositions.

In certain aspects, the present invention provides compounds having the formula, as described below:

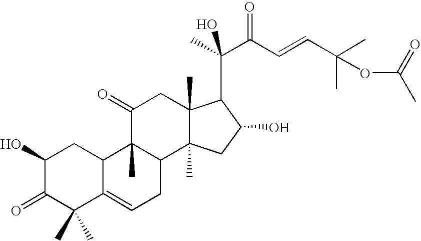

or a pharmaceutically acceptable salt, solvate, polymorph, or hydrate thereof,

The present invention also provides compositions comprising cucurbitacin B. In general, the composition is not a natural source of cucurbitacin B, such as anatomical parts of plants belonging to the genera of Cucurblta, Cucumis, Citrullus, Marah, Echinocystis, Lagenaria, Scyos, Ecballium, or Bryonia. In one aspect, a composition of the invention comprises a mixture of cucurbitacins, or pharmaceutically acceptable salts, solvates or hydrates thereof, wherein (i) the concentration of cucurbitacin B in the composition is different from that in a natural source of cucurbitacin B; and/or (ii) that the ratio of the concentration of cucurbitacin B in the composition to that of another cucurbitacin in the composition is different from that in a natural source of the cucurbitacins.

Such a composition can be prepared, for example, by processing a natural source of cucurbitacins such that cucurbitacin B has been selectively removed, retained, or enriched. Alternatively, purified cucurbitacin B can be used to make such compositions. Such a composition can also be prepared, for example, by adding an amount of cucurbitacin B to a natural source or processed form of a natural source of cucurbitacin B.

In one aspect, the present invention provides pharmaceutical compositions comprising cucurbitacin B, or a pharmaceutically acceptable salt, solvate, or hydrate thereof. In yet another aspect, the present invention also provides food additives, dietary supplements, nutraceutical compositions and food compositions comprising one or more compounds or compositions of the invention. In a specific embodiment, the pharmaceutical compositions, food additives, dietary supplements, nutraceutical compositions and/or food compositions of the invention are prepared from natural sources.

The compositions, food additives, and dietary supplements of the invention comprise an effective amount of cucurbitacin B, or pharmaceutically acceptable salts, solvates or hydrates thereof, wherein the relative amount of cucurbitacin B in the composition is different from that of a natural source. The compositions, food additives, dietary supplements, and food compositions of the invention can comprise cucurbitacin B, or pharmaceutically acceptable salts, solvates or hydrates thereof, wherein the percentages (by dry weight) of cucurbitacin B relative to the total content of cucurbitacins is different from that in a natural source of the cucurbitacins.

In yet another aspect, the invention provides methods for modulating certain signal transducing pathways that involve the activities of one or more of the following signal transducing molecules: Ras, Raf, Mek1, Mek2, Erk1, Erk2, Elk1, STAT1, STAT2, STAT3, STAT4, STAT5 and STAT6.

As the Ras-Raf-Mek-Erk-STAT signaling pathways regulate cell growth, proliferation, and death in response to external stimuli, in a specific embodiment, the compositions of the invention can be used to modulate the growth, proliferation, and death of cells in which the regulation of such pathways is disturbed or altered. Cucurbitacin B can inhibit the growth of cancer cells via the modulation of such signaling pathways in, for example, leukemia, breast cancer, prostate cancer, colon cancer, lung cancer, melanoma, liver cancer, prostate cancer, brain cancer and gastric cancer.

In yet another aspect, the invention provides methods for the prevention, treatment, management, or amelioration of a cancer or a proliferative disorder, or one or more symptoms thereof, said methods comprising administering to a subject in need thereof a prophylactically or therapeutically effective amount of the compositions of the invention. Administration of such compounds can, for example, be via one or more of the compositions, food additives, dietary supplements, nutraceutical compositions, or food compositions of the invention.

The invention also provides methods for the prevention, treatment, management, or amelioration of a proliferative disorders or a cancer, or one or more symptoms thereof, said methods comprising administering to a subject in need thereof a prophylactically or therapeutically effective amount of cucurbitacin B and a prophylactically or therapeutically effective amount of at least one other therapy (e.g., at least one other prophylactic or therapeutic agent) other than a composition of the invention. Non-limiting examples of such agents include various anti-cancer agents. Administration of such a combination of compounds can, for example, be via one or more of the compositions, food additives, dietary supplements, or food compositions of the invention.

In yet another aspect, the invention provides methods for screening natural cucurbitacin compounds or synthetic analogs, including cucurbitacin B and its derivatives, and cucurbitacin A, C, D, E, F, H, I, J, L, O, P, Q, S, T and their derivatives. The methods involve determining the activity and/or phosphorylation status of one or more of the components of a cell's signaling pathway, i.e., the phosphorylation cascade.

3.1 Terminology and Abbreviations

As used herein, “a” or “an” means at least one, unless clearly indicated otherwise. The term “about,” unless otherwise indicated, refers to a value that is no more than 10% above or below the value being modified by the term.

As used herein, the terms “disorder” and “disease” are used interchangeably to refer to an undesirable medical condition in a subject.

As used herein, the term “effective amount” refers to the amount of a compound of the invention which is sufficient to reduce or ameliorate the severity, duration of a disorder (e.g., a proliferative disorder or cancer, or one or more symptoms thereof, prevent the advancement of a disorder (e.g., a proliferative disorder or cancer), cause regression of a disorder (e.g., a proliferative disorder or cancer), prevent the recurrence, development, or onset of one or more symptoms associated with a disorder (e.g., a proliferative disorder or cancer), or enhance or improve the prophylactic or therapeutic effect(s) of another therapy.

As used herein, the term “extract” refers all possible extracts from Cucurbitaceae family, for example, but not limited to, Trichosanthes, Cucurbita pepo, Cucumis sativus and Citrullus ecirrhosus, which are obtained during the sample preparation process regardless of solvent and conditions

As used herein, the term “in combination” refers to the use of more than one therapies (e.g., one or more prophylactic and/or therapeutic agents). The use of the term “in combination” does not restrict the order in which therapies (e.g., prophylactic and/or therapeutic agents) are administered to a subject with a disorder (e.g., a proliferative disorder or an inflammatory disorder). A first therapy (e.g., a prophylactic or therapeutic agent such as a compound of the invention) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent such as an anti-inflammatory agent or anti-angiogenic agent) to a subject with a disorder (e.g., a proliferative disorder or an inflammatory disorder).

As used herein, the term “ingredient” refers to all possible products that are obtained during the sample purification process and contains lead compounds from herbs Cucurbitaceaes.

As used herein, the term “isolated” in the context of a compound such as, e.g., a compound of the invention, refers to a compound that is substantially free of chemical precursors or other chemicals when chemically synthesized. In a specific embodiment, the compound is 60%, 65%, 75%, 80%, 85%, 90%, 95%, or 99% free (by dry weight) of other, different compounds.

As used herein, the term “isolated” in the context of a compound that can be obtained from a natural source, e.g., plants, refers to a compound which is substantially free of natural source cellular material, e.g., plant cellular material, or contaminating materials from the natural source, e.g., cell or tissue source, from which it is obtained. The language “substantially free of natural source cellular material” or substantially free of plant cellular material” includes preparations of a compound that has been separated from cellular components of the cells from which it is isolated. Thus, a compound that is substantially free of cellular material (e.g., natural source cellular material, such as plant cellular material) includes preparations of a compound having less than about 30%, 20%, 10%, or 5% (by dry weight) of heterologous materials. (also referred to as a “contaminating materials”).

As used herein, the terms “manage,” “managing,” and “management” refer to the beneficial effects that a subject derives from a therapy (e.g., a prophylactic or therapeutic agent), while not resulting in a cure of the disease. In certain embodiments, a subject is administered one or more therapies (e.g., one or more prophylactic or therapeutic agents) to “manage” a disease so as to prevent the progression or worsening of the disease.

As used herein, the term “non-polar” refers to any organic solvent with a polarity index (Snyder 1978) of not greater than 2.0, and preferably not greater than 1.6. Examples of such non-polar solvents include, but are not limited to, hexane, petroleum ether, carbon tetrachloride, and mixtures of any solvents with the specified polarity index.

As used herein, the terms “non-responsive” and “refractory” describe patients treated with a currently available therapy (e.g., a prophylactic or therapeutic agent) for a disorder (e.g., a proliferative disorder or cancer), which is not clinically adequate to relieve one or more symptoms associated with such disorder. Typically, such patients suffer from severe, persistently active disease and require additional therapy to ameliorate the symptoms associated with their disorder (e.g., a proliferative disorder or cancer).

As used herein, the phrase “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylenebis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion.

As used herein, the term “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine.

As used herein, the term “polar” refers to any organic solvent with a polarity index (Snyder 1978) of greater than 2.0, and preferably greater than 4.0, and generally easily miscible with water. Examples of such moderately polar solvent include, but are not limited to, methanol, ethanol, acetonitrile, and mixtures of any solvents with the specified polarity index.

As used herein and unless otherwise indicated, the term “polymorph” means a particular crystalline arrangement of a cucurbitacin. Polymorphs can be obtained through the use of different work-up conditions and/or solvents. In particular, polymorphs can be prepared by recrystallization of a cucurbitacin in a particular solvent.

As used herein, the terms “prophylactic agent” and “prophylactic agents” as used refer to any agent(s) which can be used in the prevention of a disorder (e.g., a proliferative disorder or cancer) or one or more symptoms thereof. In certain embodiments, the term “prophylactic agent” refers to a compound of the invention. In certain other embodiments, the term “prophylactic agent” does not refer to a compound of the invention. Prophylactic agents may be characterized as different agents based upon one or more effects that the agents have in vitro and/or in vivo.

As used herein, the terms “prevent,” “preventing” and “prevention” refer to the prevention of the recurrence, onset, or development of a disorder or a symptom thereof in a subject resulting from the administration of a therapy (e.g., a prophylactic or therapeutic agents), or the administration of a combination of therapies (e.g., a combination of prophylactic or therapeutic agents).

As used herein, the phrase “prophylactically effective amount” refers to the amount of a therapy (e.g., prophylactic agent) which is sufficient to result in the prevention of the development, recurrence or onset of a disorder or a symptom thereof associated with a disorder (e.g., a proliferative disorder or cancer), or to enhance or improve the prophylactic effect(s) of another therapy (e.g., another prophylactic agent). Examples of prophylactically effective amounts of compounds are provided infra.

As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful or uncomfortable or risky. Side effects include, but are not limited to fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra pyramidal symptoms, akathisia, cardiovascular disturbances and sexual dysfunction.

As used herein, the terms “subject” and “patient” are used interchangeably herein. The terms “subject” and “subjects” refer to an animal, preferably a mammal including a non-primate (e.g., a cow, pig, horse, cat, dog, rat, and mouse) and a primate (e.g., a monkey such as a cynomolgous monkey, a chimpanzee and a human), and more preferably a human. In one embodiment, the subject is refractory or non-responsive to current treatments for a disorder (e.g., a proliferative disorder or cancer). In another embodiment, the subject is an animal that a veterinarian sees. In another embodiment, the subject is a farm animal (e.g., a horse, a cow, a pig, etc.) or a pet (e.g., a dog or a cat). In another embodiment, the subject is an animal, preferably a mammal, and more preferably a human, that is predisposed and/or at risk because of a genetic factor(s), an environmental factor(s), or a combination thereof to develop a disorder.

As used herein, the term “synergistic” refers to a combination of compounds of the invention and/or a combination of a compound or compounds of the invention and another therapy (e.g., a prophylactic or therapeutic agent), including one which has been or is currently being used to prevent, manage or treat a disorder (e.g., a proliferative disorder or cancer), which combination is more effective than the additive effects of the individual compounds or therapies. A synergistic effect of a combination of therapies (e.g., a combination of prophylactic or therapeutic agents) can permit the use of lower dosages of one or more of the therapies and/or less frequent administration of said therapies to a subject with a disorder (e.g., a proliferative disorder or cancer). The ability to utilize lower dosages of a therapy (e.g., a prophylactic or therapeutic agent) and/or to administer said therapy less frequently can reduce the toxicity associated with the administration of said therapy to a subject without reducing the efficacy of said therapy in the prevention, management or treatment of a disorder (e.g., a proliferative disorder or cancer). In addition, a synergistic effect can result in improved efficacy of agents in the prevention, management or treatment of a disorder (e.g., a proliferative disorder or cancer). Moreover, a synergistic effect of a combination of therapies (e.g., a combination of prophylactic or therapeutic agents) can avoid or reduce adverse or unwanted side effects associated with the use of either therapy alone.

As used herein, the terms “therapeutic agent” and “therapeutic agents” refer to any agent(s) which can be used in the treatment, management, or amelioration of a disorder (e.g., a proliferative disorder or cancer) or one or more symptoms thereof. In certain embodiments, the term “therapeutic agent” refers to a compound of the invention. In certain other embodiments, the term “therapeutic agent” refers does not refer to a compound of the invention. Therapeutic agents may be characterized as different agents based upon one or more effects the agents have in vivo and/or in vitro, for example, an anti-inflammatory agent may also be characterized as an immunomodulatory agent.

As used herein, the term “therapeutically effective amount” refers to that amount of a therapy (e.g., a therapeutic agent) sufficient to result in the amelioration of one or more symptoms of a disorder (e.g., a proliferative disorder or cancer), prevent advancement of a disorder (e.g., a proliferative disorder or cancer), cause regression of a disorder (e.g., a proliferative disorder or cancer), or to enhance or improve the therapeutic effect(s) of another therapy.

In a specific embodiment, with respect to the treatment of cancer, an effective amount refers to the amount of a therapy (e.g., a therapeutic agent) that inhibits or reduces the proliferation of cancerous cells, inhibits or reduces the spread of tumor cells (metastasis), inhibits or reduces the onset, development or progression of cancer or a symptom thereof, or reduces the size of a tumor. Preferably, a therapeutically effective of a therapy (e.g., a therapeutic agent) reduces the proliferation of cancerous cells or the size of a tumor by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, relative to a control or placebo such as phosphate buffered saline (“PBS”).

As used herein, the terms “therapies” and “therapy” can refer to any protocol(s), method(s), and/or agent(s) that can be used in the prevention, treatment, management, or amelioration of a disorder (e.g., a proliferative disorder or cancer) or one or more symptoms thereof. In certain embodiments, the terms “therapy” and “therapies” refer to chemotherapy, radiation therapy, hormonal therapy, biological therapy, and/or other therapies useful in the prevention, management, treatment or amelioration of a disorder (e.g., a proliferative disorder or cancer) or one or more symptoms thereof known to one of skill in the art (e.g., skilled medical personnel).

As used herein, the terms “treat”, “treatment” and “treating” refer to the reduction or amelioration of the progression, severity and/or duration of a disorder (e.g., a proliferative disorder or an inflammatory disorder), or the amelioration of one or more symptoms thereof resulting from the administration of one or more therapies (e.g., one or more therapeutic agents such as a compound of the invention). In specific embodiments, such terms refer to the inhibition or reduction in the proliferation of cancerous cells, the inhibition or reduction in the spread of tumor cells (metastasis), the inhibition or reduction in the onset, development or progression of cancer or a symptom thereof, the reduction in the size of a tumor, or the improvement in a patient's ECOG or Karnofsky score.

As used herein, the abbreviation “MAPK” refers to mitogen-activated protein kinase. The abbreviation “Erk” refers to extracellular signal-regulated kinase. The abbreviation “Mek” refers to MAPK/ERK kinase. The abbreviation “STAT” refers to signal transducer and activator of transcription.

### 4. BRIEF DESCRIPTION OF THE FIGURES

FIG. 1 depicts the growth inhibition of cucurbitacin B in 5 human leukemia cancer cell lines.

FIG. 2 depicts the apoptotic effect of cucurbitacin B on K562 cells after 48 hours treatment.

FIG. 3 depicts the apoptotic effect of cucurbitacin B on K562 cells after 48 hours treatment in flow cytometry data.

FIG. 4 depicts the change in cell morphology of K562 cells after 48 hours of cucurbitacin B treatment.

FIG. 5 depicts the cell cycle analysis profile of K562 cells after 48 hours cucurbitacin B treatment.

FIG. 6 depicts the differential activation of Stat3 in a human leukemia cancer cell line (K562) upon cucurbitacin B treatment.

FIG. 7 depicts the time dependency of Stat3 inhibition by cucurbitacin B in K562 cells.

FIG. 8 depicts cucurbitacin B inhibits activation of the Raf/Mek/Erk pathway in K562 cells but not Ras activation.

### 5. DETAILED DESCRIPTION OF THE INVENTION

The present invention relates to the inhibition of certain dysfunctional signal transduction pathways that are present in cancer cells. Signal transduction is the general process by which cells respond to extracellular signals. In typical signal transduction pathways, binding of a signaling molecule such as a hormone, neurotransmitter, or growth factor to a cell membrane receptor is coupled to the action of an intracellular second messenger. G protein-coupled receptors (GPCRs) control intracellular processes through the activation of guanine nucleotide-binding proteins (G proteins). G proteins are heterotrimeric and consist of a subunit that contains a guanine nucleotide binding domain and has GTPase activity. Hydrolysis of GTP to GDP serves as a molecular switch controlling the interactions of the subunit with other proteins.

Ras is a family of G proteins that regulate various cell functions including cell growth and differentiation, cytoskeletal organization, and intracellular vesicle transport and secretion. The Ras subfamily transduces signals from tyrosine kinase receptors, non-tyrosine kinase receptors, and heterotrimeric GPCRs (Fantl et al. (1993) Annu Rev Biochem 62:453-481). Stimulation of cell surface receptors activates Ras which, in turn, activates cytoplasmic kinases that control cell growth and differentiation. Mutant Ras proteins, which bind but do not hydrolyze GTP, are constitutively activated, and cause continuous cell proliferation and cancer (Bos (1989) Cancer Res 49:4682-4689).

The first Ras targets identified were the Raf kinases (Avruch et al. (1994) Trends Biochem Sci 19:279-283). Interaction of Ras and Raf leads to activation of the mitogen-activated protein kinase (MAP kinase) cascade of serine/threonine kinases, which activate key transcription factors that control gene expression and protein synthesis (Barbacid (1987) Ann Rev Biochem 56:779-827; Treisman (1994) Curr Opin Genet Dev 4:96-101). MAP kinases are important mediators of signal transduction from cell surfaces to nuclei via phosphorylation cascades. Several subgroups of MAP kinases have been defined and each is distinguished by a tripeptide sequence motif. The subgroups manifest different substrate specificities and responds to various distinct extracellular stimuli. (Derijard B et al (1995) Science 267:682-5). Two kinases, Mek1 and Mek2, lie directly downstream of Raf in a signaling pathway. The extracellular signal-regulated protein kinases (ERK) are characterized by Thr-Glu-Tyr, are activated by the dual phosphorylation of the threonine and tyrosine by MAP kinases located upstream of the phosphorylation cascade. The Erk/MAP kinases are phosphorylated following contact of cells with growth factors or hormones or after exposure of stress, such as heat, ultraviolet light, and inflammatory cytokines. Mutations in the signaling transducing molecules of such a phosphorylation cascade are implicated in the carcinogenesis of many different types of cancer. As shown in the example sections below, cucurbitacin B is shown to be an inhibitor of several signaling molecules of the phosphorylation cascade in the cells of a leukemic cell line.

Signal Transducer and Activator of Transcription (STAT) proteins are a class of at least six intracellular transcription factors which play an essential function in the cellular responses to cytokines (STAT1, STAT2, STAT3, STAT4, STAT5 and STAT6). These proteins contain SH2 and SH3 domains as well as a phosphorylation site at their carboxy-terminal region. After cytokine receptor activation through ligand binding, the intracellular portion of the receptor becomes phosphorylated by an associated kinase of the Janus family (JAKs). STAT proteins then bind to the phosphorylated receptor, through their SH2 domain, and are in turn phosphorylated by JAKs. Phosphorylated STAT proteins then dimerize and translocate to the nucleus, where they are able to recognize specific DNA responsive elements. Binding of the activated STAT dimer triggers transcription of the respective gene. As shown in an example below, cucurbitacin B is also an inhibitor of STAT3 in the leukemic cell line.

The invention provides methods for modulating the activities of the signal transducing pathways in animal cells, wherein the pathways involve the activities of one or more of the following signal transducing molecules: Raf, Mek1, Mek2, Erk1, Erk2, and STAT3. The inhibition of the signal transducing molecules by an effective amount of cucurbitacin B can lead to changes in the regulation of cell growth, cell proliferation and cell death, including apoptosis and necrosis as shown in the example sections. One of the contemplated pathway encompasses the following signal transducing molecules: Ras-Raf-Mek1/2-Erk1/2-STAT. As demonstrated in the examples below, a two-day treatment with cucurbitacin B inhibited the growth of cancer cell lines with a range of GI50 values. In a human leukemia cell line (K562), treatment with cucurbitacin B resulted in significant disruption of basal c-Raf, MEK and ERK phosphorylation at as early as 5 minutes after treatment and inhibited Stat3 phosphorylation 1 hour after treatment.

Many cancer cells manifest a signaling pathway involving one or more such signal transducing molecules that are mutated leading to improperly regulated signaling. Accordingly, the invention also provides methods for inhibiting the growth of cancer cells which manifest a dysfunctional signaling pathway involving one or more of the following signal transducing molecules: Raf, Mek1, Mek2, Erk1, Erk2, and STAT3. Also contemplated is the inhibition of the growth of cancer cells which comprises a mutant form of one or more of the following signal transducing molecules: Raf, Mek1, Mek2, Erk1, Erk2, Elk1 and STAT3. Non-limiting examples of such mutants include a-Raf, b-Raf and c-Raf. In addition to inhibiting the growth of such cancer cells, the invention also contemplates the induction of apoptosis in these cancer cells, and for causing necrosis of these cancer cells.

The methods of the invention described herein can be used in vitro as well as in an animal. When a certain amount of cucurbitacin B is administered to an animal comprising cells in which the signaling pathway is dysfunctional or a signaling molecule is mutated, the growth and proliferation of the cancer cells are inhibited and apoptosis is induced in the cancer cells. Accordingly, the invention provides a method of treating or preventing a proliferative disorder or a cancer, comprising administering to an animal in need thereof an effective amount of cucurbitacin B.

In another aspect of the invention, the discovery made on the mechanism of action of cucurbitacin B in cancer cells enables the development of drug screening assays based on the activity or phosphorylation status of signal transducing molecules, such as Raf, Mek1, Mek2, Erk1, Erk2, and STAT3. Accordingly, the invention provides methods for testing the therapeutic and/or prophylactic efficacies of (i) test compounds that are structurally related to cucurbitacins, including cucurbitacin A, B, C, D, E, F, H, I, J, L, O, P, Q and S, their analogs and derivatives; or (ii) test compositions comprising cucurbitacin B, or one or more cucurbitacins, said methods comprising assaying the activity of signal transducing molecules, such as Raf, Mek1, Mek2, Erk1, Erk2, and STAT3, in cancer cells in the presence or absence of the test compound or composition.

### 5.1 THE COMPOSITIONS OF THE INVENTION

The present invention provides compositions comprising cucurbitacin B, and methods of their use. The compositions and methods are described in detail in the sections below.

In one aspect, the compound have the formula as described below:

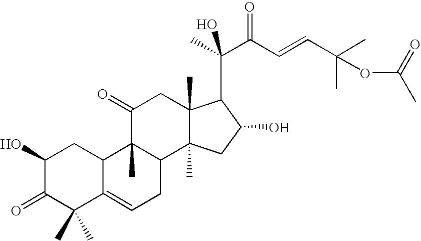

or a pharmaceutically acceptable salt, solvate or hydrate thereof,

The present invention also provides compositions comprising more than one cucurbitacins. For example, in one embodiment, a composition of the invention comprises cucurbitacin B and at least one, two, three, four, five, six, seven, eight, nine, or ten or more other cucurbitacins, such as but not limited to cucurbitacin A, cucurbitacin C, cucurbitacin D, cucurbitacin E, cucurbitacin F, cucurbitacin G, cucurbitacin H, cucurbitacin I, cucurbitacin J, cucurbitacin K, cucurbitacin 0, cucurbitacin P, cucurbitacin Q, cucurbitacin R, cucurbitacin S, cucurbitacin T, as depicted in FIG. 20.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

Accordingly, the compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

In general, the composition is not a natural source of such compounds. Examples of a natural source of such compounds include the Cucumis melo L. plant, a part of the Cucumis melo L. plant, such as the stem end, and other closely related Cucumis species and their anatomical parts. Other natural sources of such compounds include Cucurblta, Citrullus, Marah, Echinocystis, Lagenaria, Scyos, Trichosanthes, Ecballium, and Bryonia species. The term “natural source” as used herein is not limited to a plant or its anatomical part in its natural form, but is intended to include compositions or extracts which have been prepared from the plant or its parts by a process that does not selectively remove or retain cucurbitacin B relative to the other one or more particular cucurbitacins, for example, juice that is mechanically extracted from the plant, mechanically disrupted materials of the Cucumis melo L. plant or its parts, powdered stem-ends of the Cucumis melo L. plant, seed and fruits of Tricosanthes species.

In one embodiment, the composition comprises isolated cucurbitacin B. Cucurbitacins, including cucurbitacin B can also be obtained by chemical synthesis or semi-synthesis. In another embodiment, the composition comprises derivatives of cucurbitacin B, including but not limited to other non-natural tetracyclic terpenoids.

In one aspect, a composition of the invention comprises a mixture of cucurbitacins, including, cucurbitacin B or a pharmaceutically acceptable salt, solvate or hydrate thereof, wherein (i) the concentration of cucurbitacin B in the composition is different from that of a natural source of cucurbitacin B; and/or that (ii) the ratio of the concentration of cucurbitacin B in the composition to that of another cucurbitacin is different from that found in a natural source of the cucurbitacins, for example, a two-fold increase or decrease in concentration of cucurbitacin B can be used to distinguish a composition of the invention from a natural source.

Such a composition can be prepared, for example, by processing a natural source of cucurbitacins such that at least one particular cucurbitacin has been selectively removed or enriched or retained. Alternatively, purified cucurbitacin B can be used to make such compositions. Such a composition can also be prepared, for example, by adding an amount of cucurbitacin B to a natural source or prepared natural source of cucurbitacins.

As cucurbitacin B can be used in food compositions, one method for selectively removing, enriching or retaining cucurbitacins is supercritical fluid extraction. This technique, which generally utilizes carbon dioxide, is known in the art, especially for preparing food and medicinal substances for human consumption. See, for example, Hamburger et al., Phytochemical Analysis (2004), 15(1), 46-54; Simandi et al., Recents Progres en Genie des Procedes (1999) 13(71), 157-164, the disclosures of which are incorporated herein by reference in their entirety. Accordingly, in one embodiment, the invention encompasses compositions comprising cucurbitacin B that have been obtained via supercritical carbon dioxide extraction from a natural source of cucurbitacins. Such compositions are produced by a process comprising treating a natural source of cucurbitacins, such as an extract of or a stem-end of Cucumis melo L., with supercritical carbon dioxide for a period of time and at a pressure and/or a temperature that extract cucurbitacins, including conditions that selectively extract cucurbitacin B of the invention; and collecting the extracted cucurbitacins.

In another embodiment, the invention provides a composition comprising a mixture of cucurbitacins or a pharmaceutically acceptable salt, solvate or hydrate thereof, wherein the percentage (by dry weight) of cucurbitacin B relative to the total content of cucurbitacins is different from that in a natural source of the cucurbitacins. In one embodiment, the cucurbitacin B in a composition constitutes at least about 10%, at least about 20%, at least about 25%, at least about 35%, at least about 50%, at least about 75%, at least about 80%, or at least about 90% of the total cucurbitacins in the composition.

The compositions of the invention as described above can be tested by known biochemical or cell biology assays for their effect on the activity of the signaling molecules in the Ras-Raf-Mek-Erk-STAT signaling pathways, including but not limited to wild type and/or mutant forms of Raf, Mek1, Mek2, Erk1, Erk2 and STAT3. Accordingly, the invention encompasses methods for testing cucurbitacin B-containing compositions with wild type and/or mutant forms of Raf, Mek1, Mek2, Erk1, Erk2 and STAT3.

### 5.2 AGENTS USEFUL IN COMBINATION WITH THE COMPOUNDS OF THE INVENTION

The present invention provides methods for preventing, managing, treating, or ameliorating a proliferative disorder or a cancer comprising administering to a subject in need thereof a composition of the invention and one or more therapies (e.g., one or more prophylactic or therapeutic agents) other than compounds of the invention.

Any agent which contributes to the prevention, management, treatment, or amelioration of a proliferative disorder or a cancer, or one or more symptoms thereof can be used in combination with a composition of the invention in accordance with the invention described herein. See, e.g., Gilman et al., Goodman and Gilman's: The Pharmacological Basis of Therapeutics, Tenth Ed., McGraw-Hill, New York, 2001; The Merck Manual of Diagnosis and Therapy, Berkow, M. D. et al. (eds.), 17th Ed., Merck Sharp & Dohme Research Laboratories, Rahway, N.J., 1999; Cecil Textbook of Medicine, 20th Ed., Bennett and Plum (eds.), W.B. Saunders, Philadelphia, 1996 for information regarding prophylactic or therapeutic agents which have been or are currently being used for preventing, treating, managing, or ameliorating proliferative disorders or cancers or one or more symptoms thereof.

Therapeutic or prophylactic anti-cancer agents include, but are not limited to, peptides, polypeptides, fusion proteins, nucleic acid molecules, small molecules, mimetic agents, synthetic drugs, inorganic molecules, and organic molecules. Non-limiting examples of cancer therapies include chemotherapies, radiation therapies, hormonal therapies, and/or biological therapies/immunotherapies.

In certain embodiments, the anti-cancer agent is a chemotherapeutic agent. In specific embodiments, the anti-cancer agent is an anti-angiogenic agent. In other embodiments, the anti-cancer agent is not an anti-angiogenic agent. In a specific embodiment, the anti-cancer agent acts against Raf, including but not limited to sorafenib. In another embodiment, a proteasome inhibitor, such as bortezomib, is used in combination with a test composition of the invention, or the test composition with sorafenib.

Examples of anti-cancer agents include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bisphosphonates (e.g., pamidronate (Aredria), sodium clondronate (Bonefos), zoledronic acid (Zometa), alendronate (Fosamax), etidronate, ibandornate, cimadronate, risedromate, and tiludromate); bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin-2 (including recombinant interleukin 2, or rIL2), interferon alpha-2a; interferon alpha-2b; interferon alpha-n1; interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; anti-CD2 antibodies; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; Avastin®; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinyispermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; HMG CoA reductase inhibitors (e.g., atorvastatin, cerivastatin, fluvastatin, lescol, lupitor, lovastatin, rosuvastatin, and simvastatin); hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; LFA-3TIP (Biogen, Cambridge, Mass.; U.S. Pat. No. 6,162,432); liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; 5-fluorouracil; leucovorin; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; thalidomide; velaresol; veramine; verdins; verteporfin; vinorelbine; vinxaltine; vorozole; zanoterone; zeniplatin; zilascorb; and zinostatin stimalamer.

In specific embodiments, radiation therapy comprising the use of x-rays, gamma rays and other sources of radiation to destroy the cancer cells is used in combination with the antibodies of the invention. In preferred embodiments, the radiation treatment is administered as external beam radiation or teletherapy, wherein the radiation is directed from a remote source. In other preferred embodiments, the radiation treatment is administered as internal therapy or brachytherapy wherein a radioactive source is placed inside the body close to cancer cells or a tumor mass.

Cancer therapies and their dosages, routes of administration and recommended usage are known in the art and have been described in such literature as the Physician's Desk Reference (60^{th} ed., 2006).

### 5.3 USES OF THE INVENTION

Adverse health conditions, diseases and disorders which can be prevented, treated, managed, or ameliorated by administering an effective amount of cucurbitacin B or compositions of the invention include proliferative disorders and cancers, and symptoms thereof.

The compounds of the invention and compositions comprising said compounds can be used to prevent, treat, manage, or ameliorate a proliferative disorder or one or more symptoms thereof. The present invention provides methods for preventing, treating, managing, or ameliorating one or more symptoms of cellular hyperproliferation, particularly of epithelial cells (e.g., lymphoproliferative disorder), said methods comprising administering to a subject in need thereof a compound of the invention. The present invention also provides methods for preventing, managing, treating, or ameliorating a pre-cancerous disorder associated with cellular hyperproliferation, said methods comprising of administering to a subject in need thereof one or more compounds of the invention and one or more other therapies (e.g., one or more other prophylactic or therapeutic agents) useful for the prevention, treatment, management, or amelioration of said disorder. One or more of the compounds of the invention may also be used in combination with an anti-cancer therapy such as radiation therapy.

In a specific embodiment, the invention provides methods for preventing, managing, treating, or ameliorating a non-cancerous disorder associated with cellular hyperproliferation, or one or more symptoms thereof, said methods comprising of administering to a subject in need thereof a prophylactically or therapeutically effective amount of a composition of the invention.

The invention encompasses methods for preventing, treating, managing, or ameliorating one or more symptoms of a disorder associated with cellular hyperproliferation in a subject refractory to conventional therapies for such disorder, said methods comprising contacting with or administering to subject a dose of a prophylactically or therapeutically effective amount of one or more compounds of the invention. The present invention also provides methods for preventing, managing, treating, or ameliorating a non-cancerous disorder associated with cellular hyperproliferation in a subject refractory to conventional therapies for such disorder, said methods comprising of administering to a subject in need thereof one or more compounds of the invention and one or more other therapies (e.g., one or more other prophylactic or therapeutic agents) useful for the prevention, treatment, management, or amelioration of said disorder. Non-limiting examples of such prophylactic or therapeutic agents include anti-cancer agents. The compositions of the invention may also be used in combination with an anti-cancer therapy such as radiation therapy or surgery.

In another embodiment, the present invention provides methods for preventing, treating, managing, or ameliorating cancer or one or more symptoms thereof, said methods comprising administering to a subject in need thereof a composition of the invention comprising cucurbitacin B. The invention also provides methods for preventing, treating, managing, or ameliorating cancer in which one or more compounds of the invention are administered in combination with one or more other therapies (e.g., prophylactic or therapeutic agents) useful for the prevention, treatment, management, or amelioration of cancer or a secondary condition. The compositions of the invention may also be used in combination with an anti-cancer therapy such as radiation therapy or surgery.

In a specific embodiment, the invention provides a method of preventing, treating, managing, or ameliorating cancer or one or more symptoms thereof, said method comprising administering to a subject in need thereof a dose of a prophylactically or therapeutically effective amount of one or more compounds of the invention. In another embodiment, the invention provides a method of preventing, treating, managing, or ameliorating cancer or one or more symptoms thereof, said method comprising administering to a subject in need thereof a dose of a prophylactically or therapeutically effective amount of one or more compounds of the invention and a dose of a prophylactically or therapeutically effective amount of one or more therapies (e.g., one or more prophylactic or therapeutic agents) useful for the prevention, treatment, management, or amelioration of cancer, or a secondary condition (e.g., a viral, bacterial, or fungal infection).

The compounds of the invention can be used in in vitro or ex vivo for the management, treatment or amelioration of certain cancers, including leukemias and lymphomas.

One or more of the compounds of the invention may be used as a first, second, third, fourth, fifth or more line of cancer therapy. The invention provides methods for preventing, treating, managing, or ameliorating cancer or one or more symptoms thereof in a subject refractory to conventional therapies for such a cancer, said methods comprising administering to said subject a dose of a prophylactically or therapeutically effective amount of one or more compounds of the invention. A cancer may be determined to be refractory to a therapy means when at least some significant portion of the cancer cells are not killed or their cell division arrested in response to the therapy. Such a determination can be made either in vivo or in vitro by any method known in the art for assaying the effectiveness of treatment on cancer cells, using the art-accepted meanings of “refractory” in such a context. In a specific embodiment, a cancer is refractory when the number of cancer cells has not been significantly reduced, or has increased.

The invention provides methods for preventing, managing, treating or ameliorating cancer or one or more symptoms thereof in a subject refractory to existing single agent therapies for such a cancer, said methods comprising administering to said subject a dose of a prophylactically or therapeutically effective amount of one or more compounds of the invention and a dose of a prophylactically or therapeutically effective amount of one or more therapies (e.g., one or more prophylactic or therapeutic agents) useful for the prevention, treatment, management, or amelioration of cancer or a secondary condition. The invention also provides methods for preventing, treating, managing, or ameliorating cancer or a secondary condition by administering one or more compounds of the invention in combination with any other therapy(ies) (e.g., radiation therapy, chemotherapy or surgery) to patients who have proven refractory to other treatments but are no longer on this therapy(ies).

The invention provides alternative methods for the prevention, treatment, management, or amelioration of cancer where chemotherapy, radiation therapy, hormonal therapy, and/or biological therapy/immunotherapy has proven or may prove too toxic, i.e., results in unacceptable or unbearable side effects, for the subject being treated. Further, the invention provides methods for preventing the recurrence of cancer in patients that have been treated and have no disease activity by administering one or more compounds of the invention.

Cancers that can be prevented, managed, treated or ameliorated in accordance with the methods of the invention include neoplasms, tumors (malignant and benign) and metastases, or any disease or disorder characterized by uncontrolled cell growth. The cancer may be a primary or metastatic cancer. Specific examples of cancers that can be prevented, managed, treated or ameliorated in accordance with the methods of the invention include, but are not limited to, leukemia, breast cancer, prostate cancer, colon cancer, lung cancer, melanoma, liver cancer, kidney cancer, brain cancer and gastric cancer. In particular, the compositions and methods of the invention are effective against cancers with mutation in Ras and/or Raf, such as malignant melanoma, anaplastic thyroid carcinoma, papillary thyroid carcinoma, cholangiocarcinoma, colorectal carcinoma, esophageal carcinoma, acute myeloid leukemia, head and neck squamous carcinoma, non-small cell lung carcinomas, gastric carcinoma, ovarian carcinoma, mucinous ovarian carcinoma, non-Hodgkins lymphoma, renal cell carcinoma, breast carcinoma, small cell lung carcinoma, hepatocellular carcinoma, pancreatic carcinoma.

In various embodiments, lymphoproliferatve diseases that can be treated or prevented using a composition of the invention include the following: acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease

Breast cancer that can be treated or prevented using a composition of the invention include the following: adenocarcinoma, lobular (small cell) carcinoma, intraductal carcinoma, medullary breast cancer, mucinous breast cancer, tubular breast cancer, papillary breast cancer, Paget's disease, and inflammatory breast cancer

Also contemplated is the use of a composition of the invention to treat or prevent stomach cancers such as but not limited to, adenocarcinoma, fungaling (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; lung cancers such as non-small cell lung cancer, squamous cell carcinoma (epidermoid carcinoma), adenocarcinoma, large-cell carcinoma and small-cell lung cancer; prostate cancers such as but not limited to, adenocarcinoma, leiomyosarcoma, and rhabdomyosarcoma; skin cancers such as but not limited to, basal cell carcinoma, squamous cell carcinoma and melanoma, superficial spreading melanoma, nodular melanoma, lentigo malignant melanoma, acral lentiginous melanoma; and thyroid cancer such as but not limited to papillary or follicular thyroid cancer, medullary thyroid cancer and anaplastic thyroid cancer.

It is also contemplated that cancers caused by aberrations in apoptosis can also be treated by the methods and compositions of the invention.

### 5.4 COMPOSITIONS AND METHODS FOR ADMINISTRATION

The present invention provides compositions for the treatment, prophylaxis, and amelioration of proliferative disorders and cancers. Depending on the manner of use, the compositions of the invention can be a dietary supplement, a food additive, a pharmaceutical composition, or a cosmetic composition. In another embodiment, a composition of the invention comprising cucurbitacin B, or a pharmaceutically acceptable salt, solvate, polymorph, or hydrate thereof, and one or more prophylactic or therapeutic agents known to be useful for, or having been or currently being used in the prevention, treatment, management, or amelioration of a proliferative disorder or cancer, in addition to cucurbitacin B of the invention.

Generally, a dietary supplement is consumed by a subject independent of any food composition, unlike a food additive that is incorporated into a food composition during the processing, manufacture, preparation, or delivery of the food composition, orjust before its consumption. Accordingly, a food composition of the invention provides, in addition to nutrition, a therapeutic or prophylactic function to the consumer. In a specific embodiment, a composition of the invention is a food composition comprising a prophylactically or therapeutically effective amount of one or more prophylactic or therapeutic agents (e.g., a compound of the invention, and other prophylactic or therapeutic agent). In various embodiments, the composition of the invention typically comprises one or more consumable fillers or carriers. The term “consumable” means the filler or carrier that is generally suitable for, or is approved by a regulatory agency of the Federal or a state government, for consumption by animals, and more particularly by humans. In certain embodiments, the meaning of the term “dietary supplement” or “food additive” is the meaning of those terms as defined by a regulatory agency of the Federal or a state government, including the United States Food and Drug Administration.

In a specific embodiment, a composition of the invention is a pharmaceutical composition or a single unit dosage form. Pharmaceutical compositions and single unit dosage forms of the invention comprise a prophylactically or therapeutically effective amount of one or more prophylactic or therapeutic agents (e.g., a compound of the invention, or other prophylactic or therapeutic agent), and a typically one or more pharmaceutically acceptable carriers or excipients. In a specific embodiment and in this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Typical pharmaceutical compositions and dosage forms comprise one or more excipients. Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, oil, ethanol and the like. Whether a particular excipient is suitable for incorporation into a pharmaceutical composition or dosage form depends on a variety of factors well known in the art including, but not limited to, the way in which the dosage form will be administered to a patient and the specific active ingredients in the dosage form. The composition or single unit dosage form, if desired, can also contain minor amounts of wetting or emulsifying agents, or pH buffering agents.

Lactose-free compositions of the invention can comprise excipients that are well known in the art and are listed, for example, in the U.S. Pharmocopia (USP) SP (XXI)/NF (XVI). In general, lactose-free compositions comprise an active ingredient, a binder/filler, and a lubricant in pharmaceutically compatible and pharmaceutically acceptable amounts. Preferred lactose-free dosage forms comprise an active ingredient, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

The pharmaceutical compositions and single unit dosage forms can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of a prophylactic or therapeutic agent preferably in purified form, together with a suitable amount of carrier so as to provide the form for proper administration to the patient. The formulation should suit the mode of administration. In a preferred embodiment, the pharmaceutical compositions or single unit dosage forms are sterile and in suitable form for administration to a subject, preferably an animal subject, more preferably a mammalian subject, and most preferably a human subject.

A pharmaceutical composition of the invention is formulated to be compatible with its intended route of administration. Examples of routes of administration include parenteral, e.g., intravenous, intradermal, subcutaneous, oral (e.g., inhalation), intranasal, transdermal (topical), transmucosal, intra-tumoral, intra-synovial and rectal administration. In a specific embodiment, the composition is formulated in accordance with routine procedures as a pharmaceutical composition adapted for intravenous, subcutaneous, intramuscular, oral, intranasal or topical administration to human beings. In a preferred embodiment, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocamne to ease pain at the site of the injection. Examples of dosage forms include: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of inflammation or a related disorder may contain larger amounts of one or more of the active ingredients it comprises than a dosage form used in the chronic treatment of the same disease. Also, the prophylactically and therapeutically effective dosage form may vary among different types of cancer. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. Typical dosage forms of the invention comprise a compound of the invention, or a pharmaceutically acceptable salt, solvate or hydrate thereof lie within the range of from about 1 mg to about 1000 mg per day, given as a single once-a-day dose in the morning but preferably as divided doses throughout the day taken with food.

5.4.1 Oral Dosage Forms

Pharmaceutical compositions that are suitable for oral administration, and orally comsumable compositions including dietary supplements of the invention, can be presented as discrete dosage forms, such as tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include water, glycols, oils, alcohols, flavoring agents, preservatives, and coloring agents. Examples of excipients suitable for use in solid oral dosage forms (e.g., powders, tablets, capsules, and caplets) include starches, sugars, micro-crystalline cellulose, diluents, granulating agents, lubricants, binders, and disintegrating agents. Other ingredients that can be incorporated into the dietary supplement or pharmaceutical compositions of the present invention may include vitamins, amino acids, an antioxidant, a botanical extract, metal salts, and minerals.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include binders, fillers, disintegrants, and lubricants. Binders suitable for use in pharmaceutical/nutraceutical compositions and dosage forms include corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions, dietary supplements, and dosage forms disclosed herein include talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition, dietary supplement, or dosage form.

Suitable forms of microcrystalline cellulose include the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions, dietary supplmenents and dosage forms of the invention include agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions, dietary supplmenents, and dosage forms of the invention include calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions, dietary supplements, or dosage forms into which they are incorporated.

5.4.2 Delayed Release Dosage Forms

Active ingredients of the invention can be administered by controlled release means or by delivery devices that are well known to those of ordinary skill in the art. Examples include those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as tablets, capsules, gelcaps, and caplets that are adapted for controlled-release.

All controlled-release pharmaceutical products and dietary supplements have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

5.4.3 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include: Water for Injection USP; aqueous vehicles such as Sodium Chloride Injection, Ringer's Injection, Dextrose Injection, Dextrose and Sodium Chloride Injection, and Lactated Ringer's Injection; water-miscible vehicles such as ethyl alcohol, polyethylene glycol, and polypropylene glycol; and non-aqueous vehicles such as corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

5.4.4 Dietary Supplements, Food Additives, Food Compositions

The present invention provides food compositions comprising compositions and compounds of the invention. The term “food compositions of the invention” include any substances—raw, prepared or processed—which are intended for animal or human consumption, in particular by eating or drinking, and which contain nutrients in the form of carbohydrates, proteins and/or fats, and which have been modified by the incorporation of a composition, or at least one, two, three, or four compounds of the invention. A food composition of the invention provides an additional benefit other than its nutritional benefit. The present invention provides food compositions that may be used as an anti-cancer agent.

In one embodiment, a composition of the invention can be a food additive. A food additive can be in solid form or liquid form. For example, a food additive of the invention can be a reconstitutable powder that, when reconstituted with a liquid, such as drinking water, can provide a beverage. In another embodiment, a composition or compound of the invention can be incorporated into other foodstuff, such as cooking oil, frying oil, salad oil, margarine, mayonnaise or peanut butter. Oils containing the compounds of the invention can be emulsified and used in a variety of water-based foodstuffs, such as drinks. Accordingly, in one embodiment, a food composition comprising compositions and compounds of the invention can be a beverage, such as fortified mineral water, fortified distilled water, a fruit juice-based beverage, a shake, a milk-based beverage, a dairy product-based beverage, a yoghurt-based beverage, a carbonated water-based beverage, an alcoholic drink, a coffee-based beverage, a green tea-based beverage, a black tea-based beverage, a grain-based beverage, a soybean-based beverage, or a beverage based on plant extracts.

In addition to beverages, the compositions of the present invention may be used as a food additive to be combined with other foodstuff, for example, syrups, starches, grains, or grain flour. Such food composition fortified with the compounds of this invention may be used in the preparation of foodstuffs, such as baked goods, meat products with fillers (e.g., hamburgers, sausages, etc.), cereals, pastas, and soups.

The compositions or compounds of the invention can be included in food compositions which also contain a variety of other beneficial components. The optional components useful herein can be categorized by their healthful benefit or their postulated mode of action. However, it is to be understood that the optional components useful herein can in some instances provide more than one healthful benefit or operate via more than one mode of action. Therefore, classifications herein are made for the sake of convenience and are not intended to limit the component to that particular application or applications listed.

In embodiments where the compositions of the invention are dietary supplements or food additives, vitamins, precursors, and derivatives thereof, minerals, and amino acids can be added to the compositions.

The vitamins may be in either natural or synthetic form. Suitable vitamin compounds include Vitamin A (e.g., beta carotene, retinoic acid, retinol, retinoids, retinyl palmitate, retinyl proprionate, etc.), Vitamin B (e.g., niacin, niacinamide, riboflavin, pantothenic acid, etc.), Vitamin C (e.g., ascorbic acid, etc.), Vitamin D (e.g., ergosterol, ergocalciferol, cholecalciferol, etc.), Vitamin E (e.g., tocopherol acetate, etc.), and Vitamin K (e.g., phytonadione, menadione, phthiocol, etc.) compounds. The vitamins may be included as the substantially pure material, or as an extract obtained by suitable physical and/or chemical isolation from natural (e.g., plant) sources.

In another embodiment, the compounds and compositions of the invention can be added directly to foods so that an effective amount of the compound is ingested during normal meals. Any methods known to those skilled in the art may be used to add to or incorporate the compositions or compounds into natural or processed foodstuff to make the food composition of the invention. Other optional components in a food additive of the invention include anti-caking agent, dessicant, food preservatives, food coloring, and artificial sweetner.

5.4.5 Dosage & Frequency of Administration

The amount of the compound or composition of the invention which will be effective in the prevention, treatment, management, relief, or amelioration of an adverse health condition, a disorder (e.g., a proliferative disorder or cancer), or one or more symptoms thereof will vary with the nature and severity of the disease or condition, and the route by which the active ingredient is administered. The frequency and dosage will also vary according to factors specific for each subject or patient depending on the specific therapy (e.g., therapeutic or prophylactic agents) administered, the severity of the disorder, disease, or condition, the route of administration, as well as age, body, weight, response, and the past medical history of the patient. Effective doses may be extrapolated from dose-response curves derived from in vitro or animal model test systems. Suitable regiments can be selected by one skilled in the art by considering such factors and by following, for example, dosages reported in the literature and recommended in the Physician's Desk Reference (57th ed., 2003).

Exemplary doses of cucurbitacin B include milligram or microgram amounts of cucurbitacin B per kilogram of subject or sample weight (e.g., about 1 microgram per kilogram to about 50 milligrams per kilogram, about 10 micrograms per kilogram to about 5 milligrams per kilogram, or about 1 microgram per kilogram to about 20 micrograms per kilogram, or about 9 micrograms per kilogram).

In general, the recommended daily dose range of a compound of the invention for the conditions described herein lie within the range of from about 0.01 mg to about 10 mg per day, given as a single once-a-day dose preferably as divided doses throughout a day. In one embodiment, the daily dose is administered twice daily in equally divided doses. Specifically, a daily dose range should be from about 0.05 mg to about 5 mg per day, more specifically, between about 0.1 mg and about 2 mg per day. In managing the subject or patient, the therapy should be initiated at a lower dose, perhaps about 0.01 mg to about 0.25 mg, and increased if necessary up to about 2 mg to about 10 mg per day as either a single dose or divided doses, depending on the subject or patient's global response. It may be necessary to use dosages of the active ingredient outside the ranges disclosed herein in some cases, as will be apparent to those of ordinary skill in the art. Furthermore, it is noted that the dietitian, clinician or treating physician will know how and when to interrupt, adjust, or terminate therapy in conjunction with individual patient response.

Different effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the compounds of the invention are also encompassed by the above described dosage amounts and dose frequency schedules. Further, when a subject or patient is administered multiple dosages of a compound of the invention, not all of the dosages need be the same. For example, the dosage administered to the subject or patient may be increased to improve the prophylactic or therapeutic effect of the compound or it may be decreased to reduce one or more side effects that a particular subject or patient is experiencing.

In a specific embodiment, the dosage of the composition of the invention or a compound of the invention administered to prevent, treat, manage, or ameliorate a disorder (e.g., a proliferative disorder or an inflammatory disorder), or one or more symptoms thereof in a patient is about 150 μg/kg, preferably about 250 μg/kg, about 500 μg/kg, about 1 mg/kg, about 5 mg/kg, about 10 mg/kg, about 25 mg/kg, about 50 mg/kg, about 75 mg/kg, about 100 mg/kg, about 125 mg/kg, about 150 mg/kg, or about 200 mg/kg or more of a patient's body weight. In another embodiment, the dosage of the composition of the invention or a compound of the invention administered to prevent, treat, manage, or ameliorate a disorder (e.g., a proliferative disorder or an inflammatory disorder), or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

The dosages of prophylactic or therapeutic agents other than compounds of the invention, which have been or are currently being used to prevent, treat, manage, or ameliorate a disorder (e.g., a proliferative disorder or an inflammatory disorder), or one or more symptoms thereof can be used in the combination therapies of the invention. Preferably, dosages lower than those which have been or are currently being used to prevent, treat, manage, or ameliorate a disorder (e.g., a proliferative disorder or cancer), or one or more symptoms thereof are used in the combination therapies of the invention. The recommended dosages of agents currently used for the prevention, treatment, management, or amelioration of a disorder (e.g., a proliferative disorder or an inflammatory disorder), or one or more symptoms thereof can obtained from any reference in the art including Hardman et al., eds., 2001, Goodman & Gilman's The Pharmacological Basis Of Basis Of Therapeutics 10^{th} Ed, Mc-Graw-Hill, New York; Physician's Desk Reference (PDR) 60^{th} Ed., 2006, Medical Economics Co., Inc., Montvale, N.J., which are incorporated herein by reference in its entirety.

In certain embodiments, one or more compounds of the invention and one or more other the therapies (e.g., prophylactic or therapeutic agents) are cyclically administered. Cycling therapy involves the administration of a first therapy (e.g., a first prophylactic or therapeutic agents) for a period of time, followed by the administration of a second therapy (e.g., a second prophylactic or therapeutic agents) for a period of time, followed by the administration of a third therapy (e.g., a third prophylactic or therapeutic agents) for a period of time and so forth, and repeating this sequential administration, i.e., the cycle in order to reduce the development of resistance to one of the agents, to avoid or reduce the side effects of one of the agents, and/or to improve the efficacy of the treatment.

In certain embodiments, administration of the same compound of the invention may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In a specific embodiment, the invention provides a method of preventing, treating, managing, or ameliorating a disorder (e.g., a proliferative disorder or cancer), or one or more symptoms thereof, said methods comprising administering to a subject in need thereof a dose of at least 150 μg/kg, preferably at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds of the invention once every 3 days, preferably, once every 4 days, once every 5 days, once every 6 days, once every 7 days, once every 8 days, once every 10 days, once every two weeks, once every three weeks, or once a month.

5.5 Biological Assays

Based on the discovery that cucurbitacin B acts on the Ras-Raf-Mek-Erk-STAT signaling pathways in cancer cells, it is contemplated that the compositions of the invention are preferably tested using the signaling molecules in the pathway in vitro in a cell culture system, and in an animal model organism such as a rodent animal model system, for the desired therapeutic activity prior to use in humans. For example, assays which can be used to determine whether administration of a specific composition or a specific combination of therapies (e.g., a compound of the invention and an immunomodulatory agent) is indicated, include cell culture assays in which a patient tissue sample is grown in culture, and exposed to or otherwise contacted with a composition, and the effect of such composition upon the activity of one or more signaling molecules in the tissue sample or the physiology of the tissue sample is observed. The tissue sample can be obtained by biopsy from the patient. This test allows the identification of the therapeutically most effective therapy (e.g., prophylactic or therapeutic agent(s)) for each individual patient. In various specific embodiments, in vitro assays can be carried out with representative cells of cell types involved in a disorder (e.g., immune cells or cancer cells), to determine if a composition of the invention has a desired effect upon the activities of one or more signaling molecules in such cell types. As an alternative to the use of tissue, tissue samples, cancer cell lines can be used in in vitro assays. Examples of cancer cell lines that can be utilized in in vitro assays include the MCF-7 breast cancer cell line, the MCF-7/ADR multi-drug resistant breast cancer cell line, the HT114 human melanoma cell line, the MES/DOX doxorubicenresistant human uterine sarcoma cell line, the HT29 human colorectal cell line, the HCT-116 human colorectal cell line, and the K562 human leukemia cell line.

The invention provides that the compositions of the invention be assayed for their ability to modulate the activation of various types of signaling molecules and pathways, including those that are involved in the propagation of the phosphorylation cascade as well as those that lie upstream or downstream of the cascade. Activation of the pathways and factors can be determined by measuring, e.g., changes in the level of expression and/or phospharylation of cytokines, secondary messenger molecules comprising SH2 and/or SH3 domains, G proteins, tyrosine kinases, serine/threonine kinases, transcription factors and/or cell surface markers. The use of the genes and gene products of wild type and mutant forms of Ras, Raf, Mek1, Mek2, Erk1, Erk2 and STAT3 is preferred. Techniques known to those of skill in the art, including, but not limited to, immunoprecipitation followed by Western blot analysis, ELISAs, flow cytometry, Northern blot analysis, and RT-PCR can be used to measure the expression of cytokines and cell surface markers indicative of activation of the immune cell.

The compositions and compounds of the invention can be assayed for their ability to induce the expression and/or activation of a gene product (e.g., cellular protein or RNA) and/or to modulate signal transduction in cancer cells. The compositions and compounds of the invention can also be assayed for their ability to modulate the activity or phosphorylation status of the signaling molecules in the pathways. The induction of the expression or activation of a gene product or the induction of signal transduction pathways in pre-cancerous cells and/or cancer cells (in particular tubulin-binding agent resistant cancer cells) can be assayed by techniques known to those of skill in the art including, e.g., ELISAs, flow cytometry, Northern blot analysis, Western blot analysis, RT-PCR, kinase assays and electrophoretic mobility shift assays. In particular, the invention provides methods for testing the therapeutic or prophylactic efficacy of one or more cucurbitacins or a composition comprising said one or more cucurbitacins, wherein the methods comprise contacting cells with said one or more cucurbitacins and determining the activity and/or phosphorylation status of one or more signaling molecules including wild type and mutant forms of Ras, Raf, Mek1, Mek2, Erk1, Erk2 and STAT3 in the cells.

The compositions and compounds of the invention can also be assayed for their ability to modulate cell proliferation, cell growth and cell cycle progression. Techniques known to those in art, including, but not limited to, ³H-thymidine incorporation, trypan blue cell counts, and fluorescence activated cell sorting (“FACS”) analysis. The compositions of the invention can also be assayed for their ability to induce cytolysis. Cytolysis can be assessed by techniques known to those in art, including, but not limited to, ⁵¹Cr-release assays. The compositions of the invention can also be assayed for their ability to inhibit cell migration, cell adhesion angiogenesis or tubulin polymerization using techniques well-known to one of skill in the art or described herein. The compositions and compound can also be assayed for their ability to induce cell cycle arrest or apoptosis.

The compositions of the invention can be tested in suitable animal model systems prior to use in humans. Such animal model systems include rats, mice, chicken, cows, monkeys, pigs, dogs, rabbits, etc. Any animal system well-known in the art may be used. In a specific embodiment of the invention, the compositions and compounds of the invention are tested in a mouse model system. Such model systems are widely used and well-known to the skilled artisan. Pharmaceutical compositions or compounds of the invention can be administered repeatedly. Several aspects of the procedure may vary including temporal regime for administration of the compositions or compounds.

The anti-cancer activity of the compositions of the invention can be determined using any suitable animal model, including SCID mice with a tumor or injected with malignant cells. Examples of animal models for lung cancer include lung cancer animal models described by Zhang & Roth (1994, In Vivo 8(5):755-69) and a transgenic mouse model with disrupted signaling function. An example of an animal model for breast cancer includes, but is not limited to, a transgenic mouse that overexpresses cyclin D1 (see, e.g., Hosokawa et al., 2001, Transgenic Res 10(5):471-8). An example of an animal model for colon cancer includes a TCR b and p53 double knockout mouse (see, e.g., Kado et al., 2001, Cancer Res 61(6):2395-8). Examples of animal models for non-Hodgkin's lymphoma include a severe combined immunodeficiency (“SCID”) mouse (see, e.g., Bryant et al., 2000, Lab Invest 80(4):553-73) and an IgHmu-HOX11 transgenic mouse (see, e.g., Hough et al., 1998, Proc Natl Acad Sci USA 95(23):13853-8). Examples of animal models for colorectal carcinomas include Apc mouse models (see, e.g., Fodde & Smits, 2001, Trends Mol Med 7(8):369-73 and Kuraguchi et al., 2000, Oncogene 19(50):5755-63).

Further, any assays known to those skilled in the art can be used to evaluate the prophylactic and/or therapeutic utility of the compositions of the invention for the disorders disclosed herein.

The toxicity and/or efficacy of the compositions of the invention can be determined by standard pharmaceutical procedures in cell cultures or experimental animals, e.g., for determining the GI₅₀ (the growth inhibition of 50% of the population), LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/GI₅₀ or LD₅₀/ED₅₀. Compositions and compounds of the invention that exhibit large therapeutic indices are preferred. While compositions and compounds of the invention that exhibit toxic side effects may be used, care should be taken to design a delivery system that targets such compositions and compounds to the site of affected tissue in order to minimize potential damage to uninfected cells and, thereby, reduce side effects.

The data obtained from the cell culture assays and animal studies can be used in formulating a range of dosage of the compositions and compounds of the invention for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography (HPLC) and radioimmunoassay (RIA). The pharmacokinetics of a prophylactic or therapeutic can be determined, e.g., by measuring parameters such as peak plasma level (Cₘₐₓ), area under the curve (AUC, which is measured by plotting plasma concentration of the agent versus time, and reflects bioavailability), half-life of the compound (t_{1/2}), and time at maximum concentration.

Efficacy in preventing or treating a proliferative disorder such as cancer may be demonstrated, e.g., by detecting the ability of the compositions of the invention to reduce one or more symptoms of the proliferative disorder, to reduce the proliferation of cancerous cells, to reduce the spread of cancerous cells, or to reduce the size of a tumor.

### 6. EXAMPLES

In this example, the effect of cucurbitacin B on cell proliferation and apoptosis of K562 leukemia cells is elucidated. It is determined whether cucurbitacin exerts its effect via blocking of the RAS signaling pathway in K562 leukemia cells.

### 6.1 MATERIALS AND METHODS

6.1.1 Cell Lines

Human leukemia cell lines CCRF-CEM, K562, MOLT-4, RPMI-8226 and SR, purchased from the National Cancer Institute (NCI), were cultured in RPMI 1640 medium supplemented with 5% (v/v) fetal Bovine Albumin and 100 units/ml penicillin and 100 units/ml streptomycin in a humidified 5% CO₂ atmosphere at 37° C.

6.1.2 Reagents and Antibodies

Cucurbitacin B was purchased from ChromaDex, Inc. (Santa Ana, Calif.), all cell culture reagents from Gibco, Trichloroacetic acid (TCA) and Sulforhodamine B sodium salt (SRB) from Sigma, Acetic acid from Merck, Trizma base from Fluka, Annexin V-FITC Apoptosis detection kit and PI/RNase Staining Buffer from BD Pharmingen, EZ-Detect Ras Activation kit from Pierce, antibodies specific to phospho-ERK1/2, ERK1/2, phospho-MEK1/2, MEK1/2, phosphor-c-Raf, c-Raf, phosphor-Stat3, Stat3 were purchased from Cell signaling technology.

6.1.3 Cell Proliferation Assay

All 5 human leukemia cells were screened by the cell proliferation assay using SRB to quantitative cell protein mass following cucurbitacin B treatment. The experiments were repeated in triplicate. Appropriate number of cells (depends on the doubling time of cells) were seeded into a 96-well tissue culture plate and growth in the indicated medium in presence of different dosages of cucurbitacin B for 2 days. Afterwards 25 μl 80% TCA reagent was added into each well to fix the cells and the plates were incubated at 4° C. at least for 1 hour. Then the plates were washed with tap water and air dried. 100 μl 0.4% SRB reagent in Trizma-base was added to stained the cells for 10 minutes and then washed with 1% acetic acid for 4 times. The plates were air dried and dissolved by 100 μl 10 mM Trizma-base solution and the absorbance was measured at 515 nm using FLUOstar OPTIMA equipment.

6.1.4 Flow Cytometry for Apoptosis Analysis

10⁵/ml K562 cells were treated with cucurbitacin B in the presence of complete medium containing 10% FBS for 48 hours. The changes in cell morphology were visualized using Leica DMIL (Leica Microsystem). Cells were harvested and rinsed twice with ice-cold PBS (pH7.4) and a total 2.5×10⁵ K562 cells were stained with 10 μl Annexin V and 10 μl propidium iodide in 100 μl 1× binding buffer for 15 minutes in dark and then subjected to apoptosis analysis with FACSCalibar Flow cytometer system (FACS Calibur BD Flow Cytometer).

6.1.5 Flow Cytometry for Cell Cycle Analysis

K562 cells were serum starved for 24 hours prior to cucurbitacin B treatment. After 48 hours of treatment, the cells were collected and rinsed twice with phosphate-buffer saline and fixed with 80% ice-cold ethanol for 1 hour at 4° C. overnight.

Then, the cells were stained with propidium iodine at 1 mg/ml for 15 minutes at room temperature. The stained cells were analyzed by flow cytometry (FACS Calibur BD Flow Cytometer)

6.1.6 Western Blot Analysis

K562 leukemia cells were serum starved for 18 hours prior to cucurbitacin B treatment. The cells were incubated at a 75-mm culture flask filled with 10 ml growth medium (RPMI with 10% FBS and 1% PS) at the cell density of 1×10⁵ cells/ml in the presence or absence of cucurbitacin B for various time intervals. The drug treatment were terminated by centrifugation at 1500 rpm for 5 minutes and the cells were rinsed twice with phosphate-buffered saline and lysed at 4° C. in a lysis buffer containing 50 mM Tris-HCl, pH7.5, 100 μM NaCl, 5 mM EDTA, 40 mM NaP₂O₇, 1% Triton X-100, 1 mM dithiothreitol, 200 mM Na₃VO₄, 100 μM phenylmethylsulfonyl fluoride, 2 μg/ml leupeptin, 4 μg/ml aprotinin and 0.7 μg/ml pepstatin. The insoluble protein lysate were removed by centrifugation for 10 minutes at 13000 rpm. Fifteen micrograms of protein lysate was resolved using SDS-polyacryamide gel electrophoresis (PAGE) (usually 8%-12% polyacrylamide gel) and then subjected to western blot analysis. Western blots were performed with antibodies specific for phosphorylated and total Stat3, c-Raf, Mek, and Erk. The blots were developed with the Enhanced Chemiluminescence Plus (ECL Plus) detection system (Amersham).

6.1.7 GTPase Pull-Down Assay

Lysate with 500 μg protein were used to determined the Ras-GTP content by the Glutathione S-transferase (GST)-RBD (Ras-binding domain of Raf) pull down assay. The treated and control cells were lysed in lysis buffer (25 mMTris-HCl, pH7.5, 150 mM NaCl, 5 mMMgCl₂, 1% NP-40, 1 mM DTT and 5% glycerol) for 5 minutes at 4° C. Soluble cell lysates were obtained by centrifuged at 17000 g for 5 minutes. 500 μg cell lysate were collected and incubated with 80 μg GST-Raf1-RBD in 50 mM Tris-HCl, pH7.2, 150 mMNaCl, 0.5% Triton X-100, 5 mMMgCl₂, 1 mMDTT and 10% glycerol for 1 hour at 4° C. The beads were washed with lysis buffer for 3 times and then resuspended and boiled in 2× sample buffer at 100° C. for 5 minutes. 25 μl supernatants were collected by centrifuged at 7200 g for 2 minutes and then resolved in 15% SDS-PAGE followed by western blot analysis using anti-Ras antibody.

### 6.2 RESULTS AND DISCUSSION

6.2.1 Anti-Proliferation Activity of Cucurbitacin B

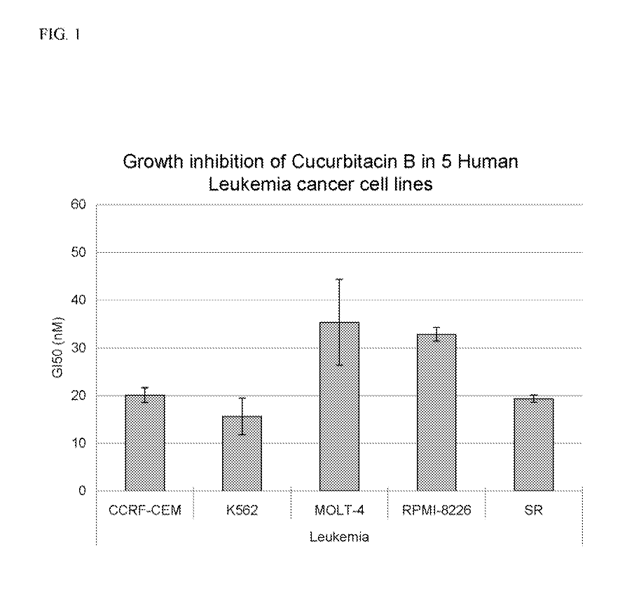

Appropriate number of cell density were seeded into a 96 well culture plate and treated with different doses of cucurbitacin B for 2 days. The growth inhibition effects of cucurbitacin B on the cells were detected using SRB. 48 hours cucurbitacin B treatment inhibited the growth of all leukemia cell lines with different GI50 values ranged from 15.6 nM to 35.3 nM, as depicted in FIG. 1. Among them, K562 had the lowest GI50 value of 15.6 nM. Introduction of a methoxy group to the ortho position of the phenol ring resulted in the significant increase of free radical scavenging activity.

6.2.2 Cucurbitacin B Induce Apoptosis and Cell Cycle Arrest in K562 Cells

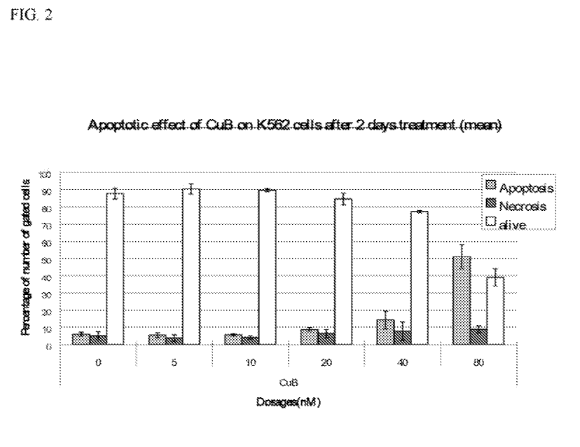

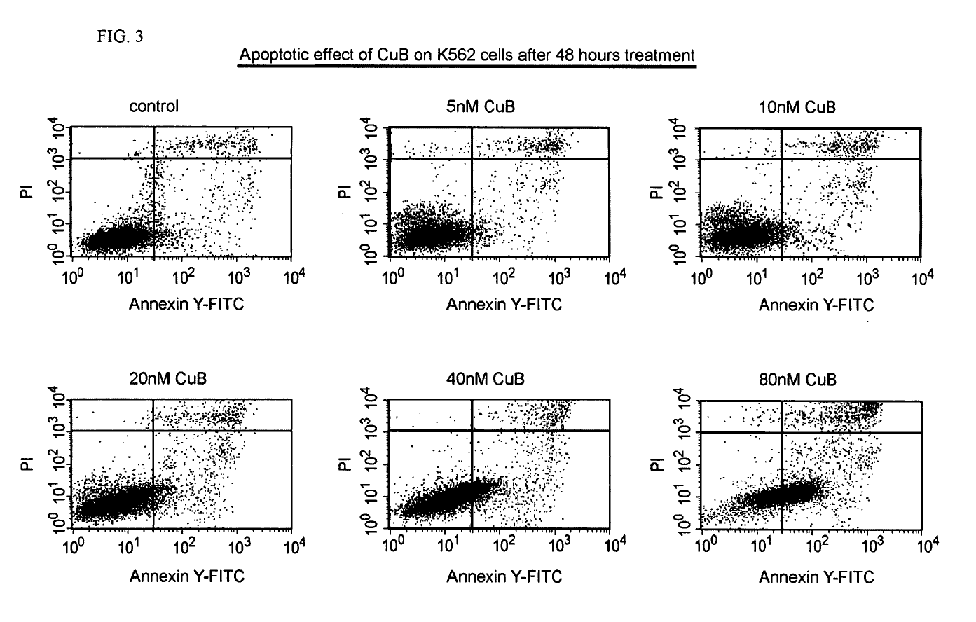

Appropriate number of cell density were seeded into T-25 culture flask and treated with different doses of cucurbitacin B for 2 days. The apoptotic effects of cucurbitacin B on the cells were detected using Flow Cytometry analysis after stained with Anx.V and PI for 15 minutes. 48 hours cucurbitacin B treatment significantly induced apoptosis in K562 cells, as depicted in FIG. 2. Cucuribitacin B induced apoptosis in a dose dependent manner, among them, 80 nM induced the highest apoptotic effect in K562 cells, with over 50% cells in apoptotic population and 10% cells in the necrotic population (late apoptotic and necrotic phase). The effect of cucurbitacin B on apoptosis and cell cycle profile in K562 cells were analyzed using flow cytometry, as depicted in FIG. 3.

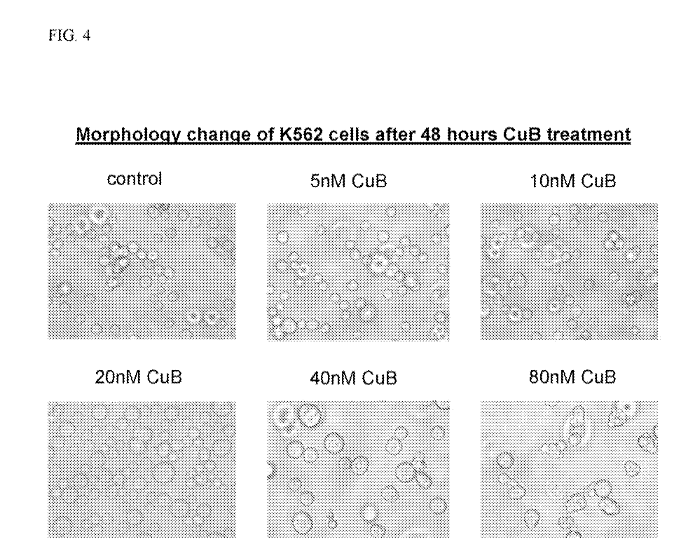

The cell morphology was significantly changed after cucurbitacin B treatment, as depicted in FIG. 4. After 48 hours of cucurbitacin B treatment, the cells were viewed using a Leica DMIL microscope at 400× magnification. Cucurbitacin B treatment significantly induced changes in cell morphorlogy, such as an increase in cell size observed in 20 nM and 40 nM-treated cells, and a shrinkage of cell size observed in 80 nM cucurbitacin B-treated cells. Enlargement of rounded cells was observed in 20 nM and 40 nM cucurbitacin B treated cells. However, the treated cells lost the rounded shape and shrank in size at the highest dose of cucurbitacin B used (80 nM).

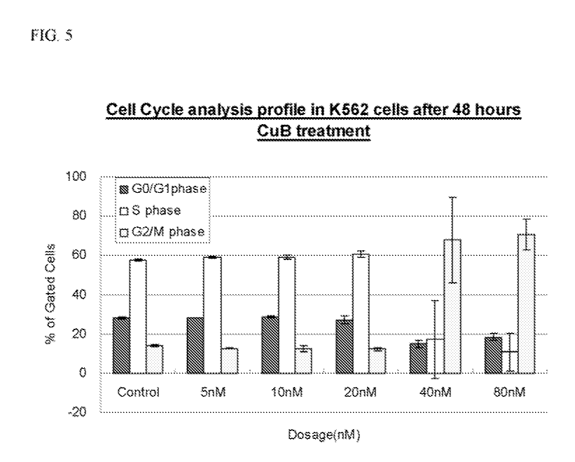

Appropriate numbers of cell density were serum starved for 24 hours prior to treatment with different doses of cucurbitacin B for 2 days. The cell cycle analysis profile of K562 cells after cucurbitacin B treatment were detected using Flow Cytometry analysis after fixed with 80% ice-cold ethanol at 4° C. and then stained with PI for 15 minutes. Cell cycle analysis showed a decreased in G1/S phase and increase of G2/M phase after 48 hours cucurbitacin B treatment in K562 cells and 80 nM cucuribitacin B induced the greater percentage of 70% in the G2/M phase, as depicted in FIG. 5.

6.2.3 Inhibition Effect Cucurbitacin B on Stat3 Activation in K562 Cells

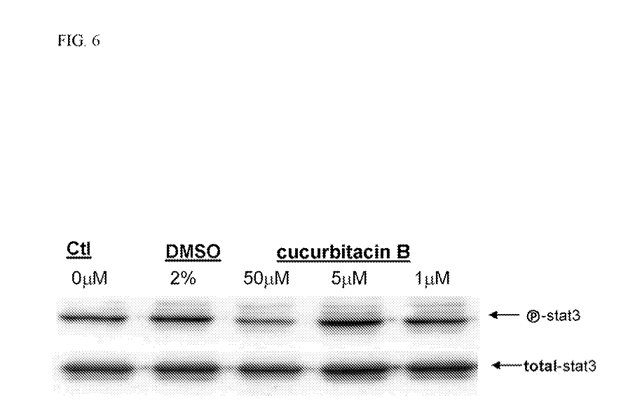

In order to determine if cucurbitacin B also demonstration in a similar mechanism in a human leukemia cells (K562) the cells were serum starve for 18 hours and were incubated either with 1 μM, 5 μM, or 50 μM cucurbitacin B for 4 hours and 2% DMSO acted as a vehicle control. 15 μg cell lysate of each sample was separated in 8% SDS-PAGE followed by immuno-blotting with anti-Stat3 antibodies to detect the phosphorylated form of Stat3 as well as its total expression level. Only the highest dose of cucurbitacin B (50 μM) significant inhibited the Stat3 activation while no significant change of Stat3 activation in the vehicle control group was observed, as depicted in FIG. 6.

To further investigate if the inhibition of cucurbitacin B on Stat3 activation was time dependent, a time course study of cucurbitacin B treatment in K562 cells was preformed. Cells were serum starved for 18 hours followed by treated with 50 μM cucurbitacin B for 10 minutes, 30 minutes, 1 hour and 4 hours, respectively. 15 μg cell lysate of each sample was separated in 8% SDS-PAGE followed by immuno-blotting with anti-Stat3 antibodies to detect the phosphorylated form of Stat3 as well as its total expression level.

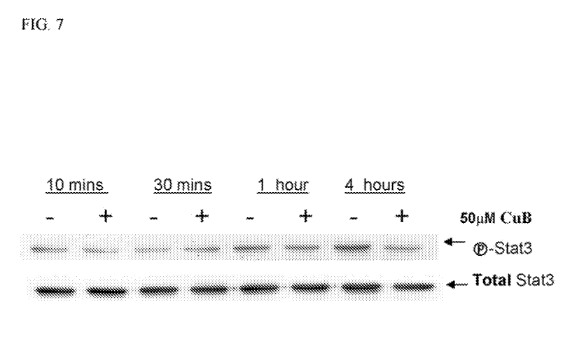

No significant change of inhibition on Stat3 activation was observed from 10 minutes to 30 minutes, however, the inhibition effect on Stat3 activation gradually increased at 1 hours and peak at 4 hours of cucurbitacin B treatment, as depicted in FIG. 7. The results show that cucurbitacin B inhibited Stat3 activation in K562 cells after 1 hour treatment.

6.2.4 Inhibition Effect of Cucurbitacin B on Raf/Mek/Erk Pathway in K562 Cells

In order to further investigate if cucurbitacin B block the Stat3 activation is truly through Erk pathway in K562 cells, the ability of cucurbitacin B to inhibit the Ras/Raf/Mek/Erk pathway was determined by measuring Ras-GTP, phosphorylation of c-Raf, Mek1/2, Erk1/2 and Elk-1 in K562 cells. The cells were incubated with 50 μM cucurbitacin B for the indicated time intervals (2 minutes, 5 minutes, 10 minutes, 30 minutes, 1 hour and 4 hours). 15 μg cell lysates were subjected to western blot analysis for phosphorylation and total c-Raf, Mek1/2, Erk1/2.

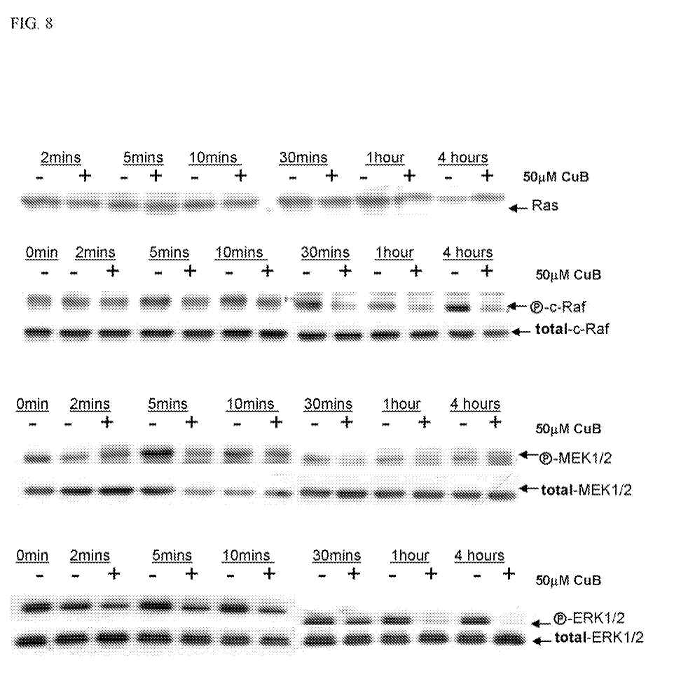

Cucurbitacin B significantly inhibited the activation of c-Raf, Mek1/2, Erk1/2 and Elk-1 upon 5 minutes treatment, as depicted in FIG. 8, while inhibition of the activation of Stat3 occurred until 1 hour after treatment. Complete inhibition of the Raf/Mek/Erk pathway was observed after 4 hours of cucurbitacin B treatment. However, no significant change of Ras-GTP activation was observed after cucurbitacin B treatment. Therefore, the results show that cucurbitacin B inhibited Raf/Mek/Erk pathway but not Ras in K562 cells, and inhibited Stat3 activation via Raf/Mek/Erk pathway in K562 cells.

### 6.3 CONCLUSION

The Ras-Raf-Mek-Erk-STAT pathway is a well-known critical singlaing pathway that medicates cell proliferation, differentiation and dealth in response to growth signals from tyrosine kinase receptor (Herrera et al., Treds Mol Med 2002; 8 (Suppl):S27-31; Schlessinger, Cell 2000; 103:211-25). Alternation of the pathway activation process is believed to lead to carcinogensis. In recent decades, its relationship with the cancer development was thoroughly investigated. Previous findings indicated that Ras mutation happened in leukemia (Ikeda et al., Mol Genet Metal. 2006; 88:216-21), colon cancer (Akagi et al., Biochem Biophys Res Commun. 2007; 352:728-32). Lung cancer (Aviel-Ronen et al., Clin Lung Cancer. 2006; 8-30-8), pancreatic cancer (Deramaudt et al., Biochim Biophys Acta. 2005; 1756:97-101), and even thyroid carcinoma (DeLellis et al., J Surg Oncol. 2006; 94:662-9). In addition, raf mutation appears more frequently in certain types of cancer. B-raf mutation is mostly found in patients with melanoma and thyroid cancer (Haluska et al., Clin Cancer Res. 2006; 12:2301s-7s; and Clampo et al., Endor Pathol. 2005; 16:163-72) while C-raf mutation/alternations is not often found in human cancer (Emuss et al, Cancer Res. 2005; 65:9719-26). Based on the ability of cucurbitacin B to inhibit the activity of these signal transducing molecules in a leukemia cell line in vitro, compositions comprising cucurbitacin B can be used to treat or prevent cancers the etiology of which implicate mutation or dysfunction of these signal transducing molecules. Further, any assays known to those skilled in the art can be used to evaluate the prophylactic and/or therapeutic utility of the compositions of the invention for the disorders disclosed herein.

All references cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

### PUBLICATIONS

Ahmad, A., Khan, K. A., Ahmad, V. U., Qazi, S., 1986 Antibacterial activity of juliflorine isolated from Prosopis juliflora. Planta Med. 4: 285-288.
Arisawa, M., Pezzuto, J. M., Kinghorn, A. D., Cordell, G. A., Farnsworth, N. R., 1984 Plant anticancer agents. XXX: Cucurbitacins from Ipomopsis aggregata (Polemoniaceae). J Pharm Sci. 73(3): 411-413.
Barbercheck, M. E. and Wang, J., 1996 Effect of cucurbitacin D on in vitro growth of Xenorhabdus and Photorhabdus spp., symbiotic bacteria of entomopathogenic nematodes. J Invertebr Pathol. 68(2): 141-145.
Bean, M. F., Antoun, M., Abramson, D., Chang, C. J., McLaughlin, J. L., Cassady, J. M., 1985 Cucurbitacin B and isocucurbitacin B: cytotoxic components of Helicteres isora. J Nat Prod. 48(3): 500.
Beutler, J. A., McCall, K. L., Herbert, K., Herald, D. L., Pettit, G. R., Johnson, T., Shoemaker, R. H., Boyd, M. R., 2000 Novel cytotoxic diterpenes from Casearia arborea. J Nat Prod. 63(5): 657-661.
Chen, J. C., Chiu, M. H., Nie, R. L., Cordell, G. A., Qiu, S. X., 2005 Cucurbitacins and cucurbitane glycosides: structures and biological activities. Nat Prod Rep. 22(3): 386-399.
Clericuzio, M., Mella, M., Vita-Finzi, P., Zema, M., Vidari, G., 2004 Cucurbitane triterpenoids from Leucopaxillus gentianeus. J Nat Prod. 67(11): 1823-1828.
Douglas, R. S., Tarshis, A. D., Pletcher, C. H. Jr., Nowell, P. C., Moore, J. S., 1995 A simplified method for the coordinate examination of apoptosis and surface phenotype of murine lymphocytes. J Immunol Methods. 188: 219-228.
Duncan, K. L., Duncan, M. D., Alley, M. C., Sausville, E. A., 1996 Cucurbitacin E-induced disruption of the actin and vimentin cytoskeleton in prostate carcinoma cells. Biochem Pharmacol. 52(10): 1553-1560.
Edery, H., Schatzberg-Porath, G., Gitter, S., 1961 Pharmaco-dynamic activity of elatericin (cucurbitacin D). Arch Int Pharmacodyn Ther. 130: 315-335.
Elledge, S. J., 1996 Cell cycle checkpoints: preventing an identity crisis. Science. 274(5293): 1664-1672.
Fang, X., Phoebe, C. H., Pezzuto, J. M. Jr., Fong, H. H., Farnsworth, N. R., Yellin, B., Hecht, S. M., 1984 Plant anticancer agents, XXXIV. Cucurbitacins from Elaeocarpus dolichostylus. J Nat Prod. 47(6): 988-993.
Fuller, R. W., Cardellina, J. H., Cragg, G. M., Boyd, M. R., 1994 Cucurbitacins: differential cytotoxicity, dereplication and first isolation from Gonystylus keithii. J Nat Prod. 57(10): 1442-1445.
Johnson, L. N., De Moliner, E., Brown, N. R., Song, H., Barford, D., Endicott, J. A., Noble, M. E., 2002 Structural studies with inhibitors of the cell cycle regulatory kinase cyclin-dependent protein kinase 2. Pharmacol Ther. 93(2-3): 113-124.
Jones, N. C., Tyner, K. J., Nibarger, L., Stanley, H. M., Cornelison, D. D., Fedorov, Y. V., Olwin, B. B., 2005 The p38α/β MAPK functions as a molecular switch to activate the quiescent satellite cell. J Cell Biol. 169(1): 105-116.
Kalejta, R. F., Shenk, T., Beavis, A. J., 1997 Use of a membrane-localized green fluorescent protein allows simultaneous identification of transfected cell s and cell cycle analysis by flow cytometry. Cytometry. 29:286-291.
Konoshima, T., Takasaki, M., Kozuka, M., Nagao, T., Okabe, H., Irino, N., Nakasumi, T., Tokuda, H., Nishino, H., 1995 Inhibitory effects of cucurbitane triterpenoids on Epstein-Barr virus activation and two-stage carcinogenesis of skin tumor. II. Biol Pharm Bull. 18(2): 284-287.
Dinan, L., Whiting, P., Girault, J. P., Lafont, R., Dhadialla, T. S., Cress, D. E., Mugat, B., Antoniewski, C., Lepesant, J. A., 1997 Cucurbitacins are insect steroid hormone antagonists acting at the ecdysteroid receptor. Biochem J. 327: 643-650.
Morgan, D. O., 1997 Cyclin-dependent kinases: engines, clocks, and microprocessors. Annu Rev Cell Dev Biol. 13: 261-291.
Musza, L. L., Speight, P., McElhiney, S., Barrow, C. J., Gillum, A. M., Cooper, R., Killar, L. M., 1994 Cucurbitacins, cell adhesion inhibitors from Conobea scoparioides. J Nat Prod. 57(11): 1498-1502.
Oberlies, N. H., Burgess, J. P., Navarro, H. A., Pinos, R. E., Soejarto, D. D., Farnsworth, N. R., Kinghorn, A. D., Wani, M. C., Wall, M. E., 2001 Bioactive constituents of the roots of Licania intrapetiolaris. J Nat Prod. 64(4): 497-501.
Pelengaris, S., Khan, M., 2003 The many faces of c-MYC. Arch Biochem Biophys. 416(2): 129-136.
Rodriguez, N., Vasquez, Y., Hussein, A. A., Coley, P. D., Solis, P. N., Gupta, M. P., 2003 Cytotoxic cucurbitacin constituents from Sloanea zuliaensis. J Nat Prod. 66(11): 1515-1516.
Setzer, W. N., Setzer, M. C., 2003 Plant-derived triterpenoids as potential antineoplastic agents. Mini Rev Med Chem. 3(6): 540-556.
Skehan, P., Storeng, R., Scudiero, D., Monks, A., McMahon, J., Vistica, D., Warren, J. T., Bokesch, H., Kenney, S., Boyd, M. R., 1990 New colorimetric cytotoxicity assay for anticancer-drug screening. J Natl Cancer Inst. 82(13): 1107-12.
Smit, H. F., van den Berg, A. J., Kroes, B. H., Beukelman, C. J., Quarles van Ufford, H. C., van Dijk, H., Labadie, R. P., 2000 Inhibition of T-lymphocyte proliferation by cucurbitacins from Picrorhiza scrophulariaeflora. J Nat Prod. 63(9): 1300-1302.
Stuppner, H. and Wagner, H. 1989 New cucurbitacin glycosides from Picrorhiza kurroa. Planta Med. 55: 559-563.
Vermes, I., Hananen, C., Steffens-Nakken, H., Reutelingsperger, C., 1995 A novel assay for apoptosis. Flow cytometric detection of phosphatidylserine expression on early apoptotic cells using fluorescein labeled Annexin V. J Immunol Meth. 184: 39-51.
Yesilada, E., Tanaka, S., Sezik, E., Tabata, M., 1988. Isolation of an anti-inflammatory principle from the fruit juice of Ecballium elaterium. J Nat Prod. 51(3): 504-508.

What is claimed is:

1. A method for inhibiting growth of cancer cells in a subject, comprising:
modulating at least one signaling molecule selected from the group consisting of Raf, Mek1, Mek2, Erk1, Erk2 and STAT3 by administering an effective amount of an isolated cucurbitacin to said subject.

2. The method of claim 1, wherein said cucurbitacin comprises cucurbitacin B, and wherein said administering an effective amount comprises contacting said cancer cells with cucurbitacin B at a concentration of 15.6 nM to 35.3 nM.

3. The method of claim 1, wherein said cancer cells are leukemia cancer cells.

4. A method for inhibiting a signal transduction pathway in cancer cells in a subject, comprising:
modulating at least one signaling molecule selected from the group consisting of Raf, Mek1, Mek2, Erk1, Erk2 and STAT3 by administering an effective amount of an isolated cucurbitacin to said subject.

5. The method of claim 4, wherein said cucurbitacin comprises cucurbitacin B, and wherein said administering an effective amount comprises contacting said cancer cells with cucurbitacin B at a concentration of 1 μM to 50 μM.

6. The method of claim 4, wherein said signal transduction pathway comprises a Raf/Mek/Erk pathway.

7. The method of claim 6, wherein said signaling molecule comprises STAT3.

8. The method of claim 6, wherein said cancer cells are leukemia cancer cells.

9. A method for inducing cell cycle arrest on cancer cells in a subject, comprising:
modulating at least one a signaling molecule selected from the group consisting of Raf, Mek1, Mek2, Erk1, Erk2 and STAT3 by administering an effective amount of an isolated cucurbitacin to said subject.

10. The method of claim 9, wherein said cucurbitacin comprises cucurbitacin B, and wherein said administering an effective amount comprises contacting said cancer cells with cucurbitacin B at a concentration of 5 nM to 80 nM.

11. The method of claim 10, wherein said cancer cells decrease in G1/S phase when contacted with cucurbitacin B at a concentration of 40 nM to 80 nM.

12. The method of claim 10, wherein said cancer cells increase in G2/M phase when contacted with cucurbitacin B at a concentration of 40 nM to 80 nM.

13. The method of claim 9, wherein said cancer cells are leukemia cancer cells.

14. A method for inducing apoptosis in cancer cells in a subject, comprising:
modulating at least one signaling molecule selected from the group consisting of Raf, Mek1, Mek2, Erk1, Erk2 and STAT3 by administering an effective amount of an isolated cucurbitacin to said subject.

15. The method of claim 14, wherein said cucurbitacin comprises cucurbitacin B, and wherein said administering an effective amount comprises contacting said cancer cells with cucurbitacin B at a concentration of 5 nM to 80 nM.

16. The method of claim 14, wherein said cancer cells increase in size when contacted with cucurbitacin at a concentration of 20 nM to 40 nM.

17. The method of claim 14, wherein said cancer cells decrease in size when contacted with cucurbitacin at a concentration of at least 80 nM.

18. The method of claim 14, wherein said cancer cells are leukemia cancer cells.
